(19) United States
(12) Patent Application Publication
(10) Pub. No.: US 2023/0234919 A1
(43) Pub. Date: Jul. 27, 2023

(54) SYNTHESIS METHOD AND APPLICATION OF METFORMIN HYDROCHLORIDE

(71) Applicant: Hainan Haili Pharmaceutical Co., Ltd., Haikou City, Hainan (CN)

(72) Inventors: Danqing XU, Haikou City, Hainan (CN); Guoxin YU, Haikou City, Hainan (CN); Liang GENG, Haikou City, Hainan (CN); Yadong ZHU, Haikou City, Hainan (CN)

(21) Appl. No.: 17/598,521

(22) PCT Filed: Sep. 14, 2021
(86) PCT No.: PCT/CN2021/118251

§ 371 Date: Sep. 27, 2021

(30) Foreign Application Priority Data
May 21, 2021 (CN) 202110561018.9

(51) Int. Cl. C07C 279/26; A61K 9/20

(52) U.S. Cl. CPC C07C 279/26; A61K 9/20

(57) ABSTRACT

The present invention belongs to the field of pharmaceutical synthesis, disclosing a synthetic process and application of metformin hydrochloride, which is to take dicyandiamine and dimethylamine, after dissolving in lower alcohol, adding sodium alkoxide, mixing evenly and gradually raising the temperature for condensation reaction, after the reaction ends, adding hydrochloric acid to adjust the pH to acidic, that is, metformin hydrochloride as described. The synthetic process of metformin hydrochloride provided in this invention, reduces the energy consumption during the reaction, reduces the production cost, and reduces the difficulty of product purification. The present invention is applicable to the synthesis of metformin hydrochloride, the as synthesized metformin hydrochloride is used to prepare metformin hydrochloride sustained release tablets.

### TECHNICAL FIELD

The present invention belongs to the field of pharmaceutical synthesis and relates to the synthesis process and application of one hypoglycemic drug class, specifically to the synthesis process and application of one metformin hydrochloride.

### BACKGROUND

Metformin hydrochloride is a white crystalline powder that is odourless. The molecular formula was C4H12ClN5 with a molecular weight of 165.62, and its structural formula was:

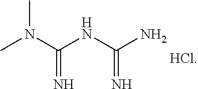

Metformin hydrochloride is a hypoglycemic agent for the treatment of type II diabetes patients with obesity, unsatisfactory dietary control alone, and ineffectiveness of physical exercise. This drug can not only lower blood glucose, but also reduce body weight, and has the effect of reducing hyperinsulinemia. It has also been shown to have clear protective cardiovascular effects and could serve as a base drug for the full course of treatment of patients with type II diabetes. In addition, as a first-line drug for the treatment of type II diabetes, compound formulations can be formed with other oral hypoglycemic agents and have good effects in the clinic. Therefore, this drug is currently the core drug for global control of diabetes.

Addition reactions are currently commercially performed at 130˜150° C. by dicyandiamide and dimethylamine hydrochloride, of which the main reaction methods are two, the melting method and the vehicle method.

Melting method, also called dry method, is a method of addition reaction after heated melting of reactants in a solvent-free system, which can better control ““triple waste”” because the reaction does not require solvents, that is, no toxic harmful reagents are introduced, and solvent recovery, liquid waste treatment and other issues need not be considered. However, due to the absence of solvents in the melt method, the mobility of the reaction is poor, the reactants are heated unevenly and the reaction is insufficient, which leads to many side reactions and greatly affects the purity and yield of the products, meanwhile, the reaction process lacks thermal control because of the absence of the use of solvents, the system is not uniform after the end of the reaction, and the operation is complicated.

The reaction process of the vehicle method uses organic solvents, as an intermediate medium does not participate in the reaction, can well dissolve dicyandiamine and dimethylamine hydrochloride, prompting the reaction to proceed homogeneously, and the heat is stable, the reaction proceeds well, and the process control is convenient; But the occurrence of the reaction requires heating to 130˜150° C., the reagents used are mostly cyclohexanol, benzene and other high boiling solvents, during the process of refining the product, not only unfriendly to the environment, but also increasing the difficulty of refining the product, the solvent can easily remain in the product affect the product purity, while increasing the amount of alcohol reagents during the refining process.

Production of metformin hydrochloride by current technologies suffers from drawbacks such as unavailability of purity, purification difficulty, high energy consumption and environmental pollution, most notably the product purity and impurity content after dicyandiamine hydrochloride and dimethylamine hydrochloride addition reaction are difficult to meet the requirements, therefore seeking a good refinement method for metformin hydrochloride is imminent.

### SUMMARY OF THE INVENTION

To solve the above deficiencies existing in the prior art, the present invention aims to provide a synthesis process of metformin hydrochloride in order to achieve the purpose of reducing the energy consumption to carry out the reaction, reducing the purification difficulty, and reducing the environmental pollution.

To accomplish the above purposes, the technical scheme employed by the present invention is as follows:

Metformin hydrochloride was synthesized by taking dicyandiamine and dimethylamine dissolved in a lower alcohol, adding sodium alkoxide, mixing well and gradually raising the temperature for condensation reaction, after the end of the reaction, after adding hydrochloric acid to adjust the pH to acidic, the said metformin hydrochloride was obtained, its reaction formula is:

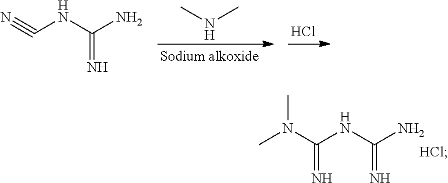

As a limit of the invention, the lower alcohols are methanol, ethanol, propanol, or ethylene glycol;

As a second qualification of the invention, the sodium alkoxide is sodium methoxide or sodium ethoxide;

As a third qualification of the invention, the gradual raising temperature is 5° C. lifting every 10 min, the reaction temperature is raised up to 80° C., and the lowest temperature of the reaction temperature is 40° C.;

As the fourth qualification of the invention, the condensation reaction is performed at a reaction temperature of 40˜80° C. and a reaction time of 2˜5 h;

As a fifth qualification of the invention, the dicyandiamide to dimethylamine molar ratio is 1:1˜1.3; The molar ratio of sodium alkoxide to dicyandiamide was 2˜2.8:1;

The invention also provides an application of a synthetic process of metformin hydrochloride for the synthesis of metformin hydrochloride; The as synthesized metformin hydrochloride was used to prepare metformin hydrochloride sustained release tablets.

As the above technical schemes are employed, the beneficial effects achieved in the present invention as compared to the prior art are:

(1) The synthetic process of metformin hydrochloride provided in this invention, by controlling the reaction rate by gradually warming, while reducing the reaction temperature, reduces the energy consumption during the reaction, and indirectly reduces the production cost;

(2) The synthetic process of metformin hydrochloride provided in this invention, since the required reaction temperature is lower, the required solvents all adopt low boiling point solvents, reducing the purification difficulty of metformin hydrochloride, the use of low boiling point solvents, prompting in the process of refining metformin hydrochloride, the solvents can be separated by drying, reducing the residue of the solvent;

(3) The present invention employs a gradual heating promotion reaction in a manner of gradually heating to proceed, since the addition of sodium alkoxide to directly warming may cause the reaction to be too vigorous, thus causing accidents such as spraying, rupture of the reaction vessel, and so on, gradually raising temperature is employed to control the reaction rate;

(4) The synthetic process of metformin hydrochloride as provided in this invention, employing solvents of low boiling point, can be recovered in such a way as to concentrate under reduced pressure that the recovered solvent can be secondarily utilized, avoiding direct abandonment of the environment from contamination.

In summary, the synthetic process of metformin hydrochloride provided in this invention reduces the energy consumption during the reaction, reduces the production cost, and reduces the difficulty of product purification.

The present invention is applicable to the synthesis of metformin hydrochloride, the as synthesized metformin hydrochloride is used to prepare metformin hydrochloride sustained release tablets.

### BRIEF DESCRIPTION OF THE FIGURES

The invention is further detailed below in combination with the drawings and specific examples.

FIG. 1 is the ion chromatogram of metformin hydrochloride A1 as synthesized in example 1 of the present invention.

### SPECIFICALLY IMPLEMENTED

The preferred examples of the invention are illustrated below in combination with the drawings. It is understood that the preferred examples described here are intended solely to illustrate and understand the invention and are not intended to qualify the invention.

### Example 1 a Synthetic Procedure for Metformin Hydrochloride A1

The present example provides a synthetic method of metformin hydrochloride A1, which is synthesized by weighing 16.8 g dicyandiamine added to 50 ml methanol dissolved, after stirring evenly, 9.1 g dimethylamine added, after stirring dissolved, 27 g sodium methoxide added, ramped 5° C. every ten minutes to the reaction temperature of 30° C., incubated and stirred for 5 h, a small amount of the reaction liquid was taken after 5 h, diluted and the reaction progress was monitored using ion chromatography, The ion peak of dicyandiamide disappeared and a new peak appeared, and the molecular weight was 130.17 (M+), this peak was the ion peak of metformin, that is, the end of the reaction, after the end of the reaction, concentrated methanol to dryness, the remaining material cooled to room temperature, 50 ml of purified water was added dropwise, 15 ml of hydrochloric add at a concentration of 38% was added to adjust the pH to 3, after mixing and stirring, another 200 ml of ethyl acetate was added, after mixing and stirring for 20 min, Layering on standing, recovery of concentrated organic phase, drying over anhydrous sodium sulfate, which can be used for the next synthesis; The obtained aqueous phase, distilled under reduced pressure until the volume of the remaining solution was one-fifth of the volume of the original aqueous phase, and the temperature of the remaining solution was reduced to room temperature, 180 mL concentration of 80% ethanol was added, mixed evenly, stop stirring, put in a freezer, low temperature crystal removal, and filtered under an ice water bath, the cake was dissolved again into 20 ml of purified water, 100 g of activated carbon was added, stirred for 10 min decolorization, After filtration, the cake was cleaned with 20 ml of purified water, 120 ml of ethanol at a concentration of 80% was added again, and after mixing was homogeneous, the crystal was crystallized at low temperature for 7 h, filtered, and the cake was dried, that is, 31.7 g of metformin hydrochloride A1 was obtained in 96.3% yield. Its reaction formula is:

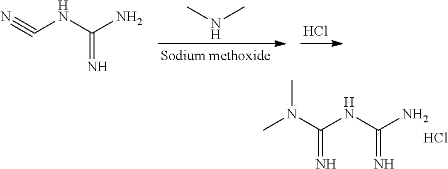

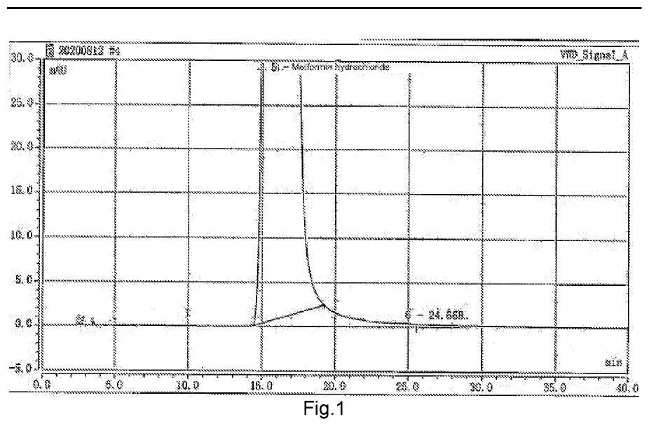

A small amount of dried metformin A1 hydrochloride was taken, diluted by dissolving in acetonitrile, and examined by ion chromatography, the purity of metformin A1 hydrochloride was 99.1%, and the obtained ion chromatogram was shown in FIG. 1.

### Example 2˜4 Synthetic Procedure for Metformin Hydrochloride A2˜A4

Examples 2˜4 provide metformin hydrochloride A2˜A4 is synthesized essentially the same as example 1 except only part of the process parameters are different, and the specific process parameters are shown in Table 1.

TABLE 1
table of specific process parameters of metformin hydrochloride A2~A4

| | | Examples | | |
|---|---|---|---|---|
| process parameters | | 2 | 3 | 4 |
| Lower alcohols (ml) | Ethanol | | 60 | |
| | Propanol | 100 | | |
| | Glycol | | | 80 |
| Sodium alkoxide (g) | Sodium methoxide | 8.1 | 30.24 | |
| | Ethanolic sodium | 17.3 | | 32.91 |
| Condensation reaction | Temperature (° C.) | 40 | 65 | 80 |
| | Time (h) | 4 | 2 | 3 |
| Dicyandiamide to dimethylamine molar ratio | | 1:1.18 | 1:1.24 | 1:1.3 |
| Molar ratio of sodium alkoxide to dicyandiamide | | 2:1 | 2.8:1 | 2.42:1 |
| Reaction pH | | 2.4 | 3 | 1.3 |
| The resulting metformin | Marked serial number | A2 | A3 | A4 |
| hydrochloride | Yield (%) | 94.5 | 93.2 | 92.8 |
| | Purity (%) | 98.9 | 99.4 | 99.0 |

All other parameters are identical to example 1.

### Synthesis of Metformin Hydrochloride by the Melt to Ratio 1 Method

Weighed 16.8 g dicyandiamine and 16.2 g dimethylamine hydrochloride into the reaction vessel, heated to 210° C., after reaction 2 h, samples were sent for inspection, the obtained test results appeared in the same position and the same molecular weight absorption peak of metformin hydrochloride in example 1, but the test map contains more impurity peaks, after the treatment of metformin hydrochloride in 82.1% yield, 95.4% purity.

### Metformin Hydrochloride was Synthesized by the Vehicle to Ratio 2

Weigh 16.8 g dicyandiamine and 16.2 g dimethylamine hydrochloride into the reaction vessel, add toluene after dissolving, heat to 150° C., after reaction for 2 h, sample for inspection, the obtained detection results appear in the same position and the same molecular weight ion peak as the ion peak of metformin hydrochloride in example 1, but the measured purity is not high, dilute after injection into the liquid chromatograph detection, The solvent peak of toluene in the obtained spectra was obvious, after cooling, concentrating, adding anhydrous ethanol crystallization, filtering, sampling for inspection, the solvent peak of toluene in the obtained results still obvious, repeatedly washed with anhydrous ethanol 5 times, each wash with anhydrous ethanol 200 ml, the toluene solvent peak in the detection results disappeared, the purity of the obtained metformin hydrochloride was 98.3%, the yield was 91.3%.

### Comparison of the Synthesis Conditions of Metformin Hydrochloride at Ratio 3 1\textcircled{ }

The synthetic process of metformin hydrochloride provided in this pair of ratios is essentially the same as example 1 except that the reaction temperature is different and the specific reaction temperature is shown in Table 2.

TABLE 2
synthetic reaction temperature table for metformin hydrochloride

| | Reaction | | Purity | |
|---|---|---|---|---|
| Sequence | Temperature (° C.) | Yield (%) | (%) | Phenomenon |
| 1 | 30 | 47.2 | 64.7 | There was more residue of raw materials and almost |
| | | | | no metformin hydrochloride in the detected spectra |
| 2 | 180 | 80.3 | 94.5 | Solvent reduction was rapid, side reactions |
| | | | | increased, and impurity peaks increased in the |
| | | | | detection profiles |

### Versus Synthetic Conditions for Ratio 4 Metformin Hydrochloride \textcircled{2}

The synthetic procedure for metformin hydrochloride as provided in this pair of ratios is essentially the same as example 1 except that the synthesis is carried out by heating directly (i.e., rapidly or at a rate of 1° C./min or higher) to 70° C. during the heating process, a large number of bubbles are generated in the reaction liquid, and part of the reaction liquid spills out from the mouth of the reaction vessel, meanwhile a thermometer shows that the reaction liquid has risen rapidly, After explosion-proof plate was added, the reaction was closed after 2 h, and after the reaction liquid was cooled and no more vigorous bubbling, samples were sent for examination, and no metformin hydrochloride was produced in the resulting patterns.

### Preparation of Sustained Release Tablets of Metformin Hydrochloride for Application

Metformin hydrochloride sustained release tablets were prepared by randomly selecting one of the synthesized metformin hydrochloride A1˜A4 from examples 1 to 4, and the specific preparation was as follows:

### 1) Crushing Sieving

Metformin hydrochloride, sodium carboxymethyl cellulose were crushed through an 80 mesh sieve before dosing production; Hydroxypropyl methylcellulose k100m, microcrystalline cellulose, hydroxypropyl methylcellulose E5 through a 60 mesh sieve, the rest of the material was directly weighed for use.

### 2) Granulation

Mixing: add the sieved metformin hydrochloride, sodium carboxymethyl cellulose to the boiling dry granulator, fan set 35 Hz, do not open heat, and mix for 10 min.

Granulation: set the fan frequency to 35 Hz, inlet temperature 80.0° C., set the atomization pressure 0.35 MPa for the inner layer and 0.30 MPa for the outer layer (or 0.05 MPa larger for the inner layer but not less than 0.2 MPa for the outer layer), set the feed rate 300 R/min, and start the spray granulation when the material temperature reaches 45° C.±5° C. and lift it every 5 min for 25 R. When the feed frequency reaches 450 R/min, until the end of the water spray and the material temperature reaches 42.0±2° C. then the pan drops to observe the material granulation. The total amount of water spray used in granulation here was 85 kg.

Second spray: without turning on hot air, set the fan frequency at 40 Hz and the water supply frequency at 500 R/min to spray wet the material. The fan frequency determines the drying rate, and the supply frequency determines the spray rate. The total amount of water to be sprayed on the second time is ¼ ˜½ of the amount to be sprayed on mixing the pellets.

Mixing: draw ⅓ of the prescribed amount of hydroxypropyl methylcellulose k100m into a boiling dry granulator and mix to dry for 5-10 min.

Whole grain: charge prescribed amount of silica into the mass compartment for whole grain using a lifting flip integrator with a 1.5 mm screen installed by the whole grain, push the boiling dryer's bin under the dry integrator, tighten the 6 coupling screws of the coupling barrel cover and seal after tightening; After pushing the bin cart away, operating the elevator flips the bucket 180° and rises to the appropriate height. Advance the 1000 l mixing tank so that the mixing bucket outlet is aligned to the inlet of the dry homogenizer, the outlet of the dry homogenizer is aligned to the mixing tank orifice and secured with a hose. Start the pellet. Set the whole grain rotation rate at 10 Hz. Open the barrel lid outlet knob and perform a full pelletization. After the end of the whole pellet ejection, close the barrel cap outlet knob and stop the whole pellet.

### 3) Total Admixture

Open the mixer hopper cover and add the remaining prescribed amounts of hydroxypropyl methylcellulose k100m, hydroxypropyl methylcellulose E5, microcrystalline cellulose and magnesium stearate into the mass chamber, pour the whole completed pellets, close and pinch the hopper cover, set the mixing time to 25 min, the mixing speed to 10 R/min and press the blend button to start mixing. After mixing, the intermediate pellet was sent for inspection at the fill port sampling volume.

### 4) Press

Formulate the stress tablet weight according to the content of intermediate granules, install 19 mm*9 mm metformin hydrochloride sustained release tablets special shape punch for tableting, tablet hardness is controlled at 170n˜200N, friability should not exceed 0.8%.

### 5) Blister Packing

Select 8/plate or 10/plate molds for packaging. Collect the intermediate product, PVC and aluminum foil, install the forming mold, heat sealing mold, print mold, punch planting mold, set up the fully automatic high-speed blister packaging machine air pressure 0.6 MPa˜0.8 MPa, upper and lower forming mold temperature 120˜140° C., heat sealing temperature 200° C.˜240° C., 20˜45 Hz punch speed, for packaging, i.e., metformin hydrochloride sustained release tablets M.

Stability Test of Extended Release Form of Metformin Hydrochloride in Experimental Cases

According to the method of preparing metformin hydrochloride sustained release tablets provided in the application example, D1, D2 of the synthesized metformin hydrochloride sustained release tablets in ratios 1-2; Metformin hydrochloride sustained release tablets (Qingdao Yellow Hai Pharmaceutical Co., Ltd.) were procured as metformin hydrochloride sustained release tablets D3

Test methods: stability test inspections were performed with reference to the second part of the Chinese Pharmacopoeia, 2015

Test conditions:

Long term test temperature: 25±2° C.;

Long term test humidity: rh60±10%;

Long term trial study duration: 0, 3, 6, 9, 12, 18, 24 months

The test results are shown in Table 3.

TABLE 3
Table of results of stability test of sustained release tablets of metformin hydrochloride.

| | Metformin | Metformin | Metformin | Metformin |
|---|---|---|---|---|
| | hydrochloride | hydrochloride | hydrochloride | hydrochloride |
| | sustained release | sustained release | sustained release | sustained release |
| | tablets M | tablets D1 | tablets D2 | tablets D3 |
| Month | Impurity content (%) | Impurity content (%) | Impurity content (%) | Impurity content (%) |
| 0 | 0.61 | 4.6 | 1.7 | 0.81 |
| 3 | 0.63 | 4.65 | 1.76 | 0.83 |
| 6 | 0.67 | 4.69 | 1.79 | 0.86 |
| 9 | 0.71 | 4.71 | 1.82 | 0.88 |
| 12 | 0.73 | 4.74 | 1.86 | 0.9 |
| 18 | 0.75 | 4.78 | 1.90 | 0.93 |
| 24 | 0.77 | 4.86 | 2.02 | 0.95 |
| Total increment | 0.16 | 0.26 | 0.32 | 0.14 |
| of impurities | | | | |
| (%) | | | | |

Our results showed that metformin hydrochloride sustained-release tablets prepared from metformin hydrochloride by using the synthesis method provided in the present work exhibited a small impurity increment of only 0.16% during 24 months of standing, which was superior to metformin hydrochloride sustained-release tablets D1 and D2 and similar to the stability of commercially available metformin hydrochloride sustained-release tablets D3.

It is required that said above be used only for preferred examples of the invention and are not intended to restrict the invention, although the invention is elaborated with reference to the above examples, which for those skilled in the art can still be modified with respect to the technical schemes documented for each of the above examples, or with equivalent replacement for portions of the technical features there in. Any modifications, equivalent replacements, refinements, etc. made shall be included within the spirit and principles of the invention.

1. A synthetic method of metformin hydrochloride, The synthesis is to take dicyandiamine and dimethylamine dissolved in lower alcohol, add sodium alkoxide, after mixing evenly gradually raising the temperature for condensation reaction, after the end of the reaction, after adding hydrochloric acid to adjust the pH to acidic, that is, to obtain the said metformin hydrochloride, the reaction formula is:

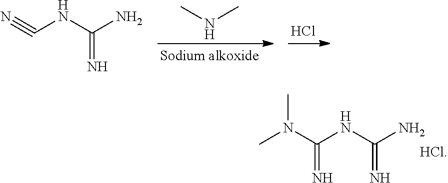

2. A synthetic method of metformin hydrochloride as recited in claim 1, The described gradual raising temperature was 5° C. lifting per 10 min interval, the highest raising of the reaction temperature was up to 80° C., and the lowest raising temperature of the reaction temperature was 20° C.

3. A synthetic method of metformin hydrochloride as recited in claim 2, The lower alcohols are methanol, ethanol, propanol or glycol; The sodium alkoxide is sodium methoxide or sodium ethoxide.

4. A synthetic method of metformin hydrochloride as recited in claim 1, The reaction temperature for the condensation reaction ranged from 40 to 80° C. and the reaction time ranged from 2 to 5 h.

5. A synthetic method of metformin hydrochloride as recited in claim 1, The dicyandiamide to dimethylamine molar ratio was 1:1˜1.3; The molar ratio of sodium alkoxide to dicyandiamide was 2˜2.8:1; The pH adjustment is utilizing hydrochloric acid to adjust the pH value to 1˜3.

6. One application of the synthetic process of metformin hydrochloride according to claim 1, the synthetic procedure described is for the synthesis of metformin hydrochloride; The as synthesized metformin hydrochloride was used to prepare metformin hydrochloride sustained release tablets.

7. A synthetic method of metformin hydrochloride as recited in claim 3, The reaction temperature for the condensation reaction ranged from 40 to 80° C. and the reaction time ranged from 2 to 5 h.

8. A synthetic method of metformin hydrochloride as recited in claim 2, The dicyandiamide to dimethylamine molar ratio was 1:1˜1.3; The molar ratio of sodium alkoxide to dicyandiamide was 2˜2.8:1; The pH adjustment is utilizing hydrochloric acid to adjust the pH value to 1˜3.

9. A synthetic method of metformin hydrochloride as recited in claim 3, The dicyandiamide to dimethylamine molar ratio was 1:1˜1.3; The molar ratio of sodium alkoxide to dicyandiamide was 2˜2.8:1; The pH adjustment is utilizing hydrochloric acid to adjust the pH value to 1˜3.

10. One application of the synthetic process of metformin hydrochloride according to claim 2, the synthetic procedure described is for the synthesis of metformin hydrochloride; The as synthesized metformin hydrochloride was used to prepare metformin hydrochloride sustained release tablets.

11. One application of the synthetic process of metformin hydrochloride according to claim 3, the synthetic procedure described is for the synthesis of metformin hydrochloride; The as synthesized metformin hydrochloride was used to prepare metformin hydrochloride sustained release tablets.

12. One application of the synthetic process of metformin hydrochloride according to claim 4, the synthetic procedure described is for the synthesis of metformin hydrochloride; The as synthesized metformin hydrochloride was used to prepare metformin hydrochloride sustained release tablets.

13. One application of the synthetic process of metformin hydrochloride according to claim 5, the synthetic procedure described is for the synthesis of metformin hydrochloride; The as synthesized metformin hydrochloride was used to prepare metformin hydrochloride sustained release tablets.
